(19) United States
(12) Patent Application Publication
(10) Pub. No.: US 2010/0183768 A1
(43) Pub. Date: Jul. 22, 2010

(54) TREATMENT OF CELL SUSPENSION

(76) Inventors: Karsten Baaner, Slangerup (DK); Mette Winning, Frederiksberg C (DK); Peter Wagner, Hoersholm (DK)

Correspondence Address: FOLEY AND LARDNER LLP;SUITE 500, 3000 K STREET NW, WASHINGTON, DC, 20007, US

(21) Appl. No.: 12/664,957

(22) PCT Filed: Jun. 27, 2008
(86) PCT No.: PCT/EP2008/058322

§ 371 Date: Feb. 4, 2010

(30) Foreign Application Priority Data
Jun. 28, 2007 (DK) PA 2007 00939

(51) Int. Cl. C12N 1/20; A23L 1/30; A23K 1/16

(52) U.S. Cl. 426/61; 435/252.9

(57) ABSTRACT

A new method for making a composition comprising a particle containing cell culture (e.g. a lactic acid bacteria culture) suspended in oil, a lipid, a wax or a mixture of those, wherein the composition gives improved storage stability of the cell of interest.

### FIELD OF THE INVENTION

The present invention relates to a new method for making a composition comprising a particle containing cell culture (e.g. a lactic acid bacteria culture) suspended in oil, a lipid, a wax or a mixture of those, wherein the composition gives improved storage stability of the cell of interest.

### BACKGROUND OF THE INVENTION

Cells such as e.g. microorganisms are involved in numerous industrially relevant processes. For instance bacterial cultures, in particular cultures of bacteria that are generally classified as lactic acid bacteria are essential in the making of all fermented milk products, cheese and butter. Cultures of such bacteria may be referred to as starter cultures and they impart specific features to various dairy products by performing a number of functions.

Storage stability of commercially relevant microorganism compositions (e.g. LAB starter cultures) are commercially important and the art describes a number of different technical solutions to this (e.g. addition of different cryoprotective agents).

In the present context, WO2004/028460 (Probiohealth LLC) is considered relevant. It describes an oil emulsion/suspension comprising LAB probiotic bacteria. The suspension is made by simply mixing a LAB composition (e.g. a freeze-dried powder) with oil to get the suspension (see e.g. example 1).

WO2004/028460 describes a “standard” way of making an oil suspension comprising microorganisms, which in short may be described as the simple mixing together of microorganism and oil. At the filing date of the present application the present inventors were not aware of prior art documents that describe other relevant more “sophisticated” methods for making such oil-microorganism suspensions.

### SUMMARY OF THE INVENTION

The problem to be solved by the present invention relates to the provision of a new method to make a composition comprising a cell (e.g. a LAB) suspended in oil, a lipid or a wax. As illustrated in working examples herein, the new composition made by the new method as described herein gives an improved storage stability of the cell of interest.

The solution of the present invention is based on a method, wherein e.g. a cell-oil suspension is put under vacuum.

Without being limited to theory is below provided a description of what is believed to happen by involvement of the vacuum treatment as described herein. For illustration, reference is made to FIG. 1 herein, which illustrates an example of the method of the invention. Reference is also made to working example 1 herein, wherein it is demonstrated that storage stability of the LAB cell L. acidophilus was significantly improved in a composition made by a process involving the herein described vacuum step. Similar significant storage stability improvement was also demonstrated for a cell of a different genus Bifidobacterium lactis (see working example 5 herein).

A commercially relevant microorganism culture may be a freeze-dried culture in the form of a powder. This powder comprises many individual particles. Each particle of the powder is a porous structure comprising the microorganism of interest plus other material and compounds generally derived from the previous fermentation process.

Step (a) of FIG. 1 illustrates that the first step, of an example of a method as described herein, is to make an oil-powder suspension (e.g. by simply mixing the oil and microorganism powder). As explained above the particles of the powder are porous. Within the particles there are micro pockets of gas.

In step (b) of FIG. 1, the oil-powder suspension is put under vacuum. The effect of this is that at least a significant part of the gas within the particles is removed. As illustrated in working examples herein this can be observed as bubbles of gas escaping from the oil-powder suspension.

In step (c) of FIG. 1, the vacuum is removed. Within the suspension, the individual particles are covered by oil. The particles have a lot of “empty” pockets, which before vacuum were “occupied” by gas. Accordingly, when vacuum is removed the oil will rapidly enter and fill out these pockets. The final result is that the micro pockets originally filled with gas are now filled with oil and one therefore gets a suspension, wherein each particle containing microorganisms comprises significantly less gas.

As illustrated in working examples herein, the new suspensions as described herein result in an improved storage stability of the cell. A theory behind this positive effect relates to the fact that the vacuum step removes the gas from the micro pockets within the cell powder particles. If the gas is not removed during the process of the oil encapsulation, then the gas will allow for rapid transport of moisture, oxygen or other components that are transported more rapidly through gas compared to through the oil. A rapid transport of these components through the encapsulated product will decrease the stability, especially when the product surface area is high as compared to the product mass or volume.

Oil is a very suitable material to be used as described herein. However, other materials that provide a poor transport of moisture, oxygen or other components that can damage the viability of the microorganisms directly or indirectly may also be used. Besides oil such suitable materials include wax or lipid.

Accordingly, the invention relates to a method for making a composition comprising a cell culture suspended in oil, a lipid, a wax or a mixture of those comprising the following steps:
(a): mixing a cell culture comprising porous particles containing cells with a material comprising oil, a lipid, a wax or a mixture of those;
(b): creating a vacuum over the suspension; and
(c): removing the vacuum over the suspension.

In a first aspect the invention relates to a method for making a composition comprising a cell culture suspended in oil, a lipid, a wax or a mixture of those comprising following steps:
(a): mixing a cell culture comprising porous particles containing cells into a material comprising oil, a lipid, a wax or a mixture of those to get a suspension;
(b): creating a vacuum over the suspension in order to remove a suitable amount of the gas, present within the porous particles, from the suspension; and
(c): removing the vacuum over the suspension to get an adequate pressure allowing the oil, the lipid, the wax or a mixture of these, that cover the particles, to enter into the porous particles and thereby occupy a suitable amount of the space within the particles that before the vacuum step (b) were occupied by gas;

to get a composition comprising a cell culture suspended in oil, a lipid, a wax or a mixture of those characterized by that a significant amount of the space within the porous particles containing cells that before the vacuum step (b) were occupied by gas are occupied by oil, a lipid, a wax or a mixture of those.

The composition that can be obtained by a method of the first aspect is in itself a novel composition. Compared to similar e.g. oil-bacteria compositions of the prior art (see e.g. WO2004/028460 discussed above) the novel composition as described herein is different in the sense that a suitable amount of the space within the porous particles containing cells that before the vacuum step (b) were occupied by gas are occupied by oil, a lipid, a wax or a mixture of those. Since the methods described in the prior art do not involve the vacuum step the composition described in the prior art cannot be identical to the compositions of the present invention. The fact that there is a “structural” difference of the composition as described herein over similar prior art compositions is also implicitly demonstrated by the fact the compositions of the invention gives improved storage stability of the cell as compared to control compositions made without involvement of the vacuum step.

Accordingly, a second aspect of the invention relates to a composition comprising a cell culture suspended in oil, a lipid, a wax or a mixture of those obtainable by a method of the first aspect of the invention and embodiment thereof as described herein and characterized by that a significant amount of the space within the porous particles containing cells that before the vacuum step (b) of the first aspect were occupied by gas are occupied by oil, a lipid, a wax or a mixture of those.

### DRAWING

FIG. 1: Illustration of a method for making a composition comprising a microorganism suspended in oil as described herein. The method includes the vacuum step.

### DETAILED DESCRIPTION OF THE INVENTION

In a first aspect, the invention relates to a method for making a composition comprising a cell culture suspended in oil, a lipid, a wax or a mixture of those comprising the following steps:
(a): mixing a cell culture comprising porous particles containing cells with a material comprising oil, a lipid, a wax or a mixture of those;
(b): creating a vacuum over the suspension; and
(c): removing the vacuum over the suspension.

Embodiments of the method of the invention are:
A method wherein the cell is a lactic acid bacteria (LAB), preferably wherein the lactic acid bacteria is Lactobacillus acidophilus, and wherein the composition comprising a cell culture has a content of viable cells of at least 10⁶ colony forming units (CFU) per g composition;
A method wherein the cell culture comprising porous particles containing cells of step (a) is a freeze-dried culture in the form of a powder;
A method wherein the material to be mixed with the cell culture of step (a) of the method of the invention is oil, preferably a vegetable oil selected from the group consisting of: hazelnut oil, olive oil, primrose oil, pumpkin oil, rice-bran oil, soybean oil, maize oil and sunflower oil;
A method wherein the mixture of step (a) is stirred until no visible lumps are detected in the suspension;
A method wherein the suspension of step (a) comprises from 5 to 40% of the cell culture and from 60 to 95% of the relevant material and wherein the sum of the two components cell culture and relevant material amount to at least 95% of the suspension;
A method wherein the vacuum treatment of step (b) is maintained until there is virtually no gas bubbles escaping from the suspension;
A method wherein a viscosity enhancer is added to the composition after the vacuum removal step (c) in order to get a composition with a viscosity of interests;
A method wherein the composition comprising the cell culture obtained after step (c) is packed into a suitable capsule; and/or
A method wherein the composition comprising the cell culture obtained after step (c) is sprayed onto the surface of cereals.

In a second aspect, the invention relates to composition obtainable by a method of the invention. A presently interesting composition comprises a Bifidobacterium strain and a vegetable oil, ie a composition which comprises a strain selected from the group consisting of: BB-12®, ATCC 29682, ATCC 27536, DSM 13692, DSM 15954, and DSMZ 10140, and an vegetable oil, preferably selected from the group consisting of: hazelnut oil, olive oil, primrose oil, pumpkin oil, rice-bran oil, soybean oil, maize oil and sunflower oil.

The aspect embraces a composition obtainable by a method comprising the following steps:
(a): providing a suspension containing (i) cells, (ii) porous particles, and (iii) an oil, a lipid, or a wax;
(b): creating a vacuum over the suspension; and
(c): removing the vacuum over the suspension.

The composition of the invention may be encapsulated, e.g. in a gelatin capsule.

In further aspects, the invention relates to a food product, such as a cereal, a dairy product, or a juice, comprising a composition of the invention, and to a food or feed additive comprising a composition of the invention.

The Cell

The cell may in be principle be any suitable cell of interest such as any eukaryotic or prokaryotic cell. Preferably the cell is a cell selected from the group consisting of a filamentous fungal cell and a microorganism cell.

Preferably, the cell is a probiotic cell. This is particularly preferred when the cell is a lactic acid bacterium (see below).

The expression “probiotic cell” designates a class of cells (e.g. micro-organisms) which is defined as a microbial food or feed supplement which beneficially affects the host human or animal by improving its gastrointestinal microbial balance. The known beneficial effects include improvement of the colonization resistance against the harmful micro-flora due to oxygen consumption and acid production of the probiotic organisms. An example of the efficacy of probiotically active organisms to prevent overgrowth of potential pathogens and thus diarrhea, is shown in a study where the administration of capsules containing viable probiotically active organisms to tourists traveling in Egypt resulted in a protection rate of 39.4% against traveler's diarrhea (Black et al. 1989). A review of probiotics and their effects in man and animals can be found in Fuller, 1989 and 1994.

Filamentous fungi include all filamentous forms of the subdivision Eumycota and Oomycota (as defined by Hawksworth et al., 1995, supra). The filamentous fungi are characterized by a vegetative mycelium composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In a more preferred embodiment, the filamentous fungal cell is a cell of a species of, but not limited to, Acremonium, Aspergillus, Fusarium, Humicola, Mucor, Myceliophthora, Neurospora, Penicillium, Thielavia, Tolypocladium, and Trichoderma or a teleomorph or synonym thereof.

A preferred microorganism cell suitable to be used in a method as described herein is a microorganism cell selected from the group consisting of yeast cells and prokaryotic cells.

A preferred yeast cell is a yeast cell selected from the group consisting of Ascomycetes, Basidiomycetes and fungi imperfecti. Preferably a yeast cell selected from the group consisting of Ascomycetes.

Preferred Ascomycetes yeast cells are selected from the group consisting of Ashbya, Botryoascus, Debaryomyces, Hansenula, Kluveromyces, Lipomyces, Saccharomyces spp e.g. Saccharomyces cerevisiae, Pichia spp., Schizosaccharomyces, spp.

A preferred yeast cell is a yeast cell selected from the group consisting of Saccharomyces spp e.g. Saccharomyces cerevisiae, and Pichia spp.

In a method as described herein a very preferred cell is a prokaryotic cell. A preferred prokaryotic cell is selected from the group consisting of Bacillus, Streptomyces, Corynebacterium, Pseudomonas, lactic acid bacteria and an E. coli cell.

A preferred Bacillus cell is B. subtilis, B. amyloliquefaciens or B. licheniformis.

A preferred Streptomyces cell is S. setonii.

A preferred Corynebacterium cell is C. glutamicum.

A preferred Pseudomonas cell is P. putida or P. fluorescens

Commercial dairy starter cultures are generally composed of lactic acid and citric acid fermenting lactic acid bacteria. Accordingly, in a very preferred embodiment the cell is a lactic acid bacterium (LAB).

In the present context, the expression “lactic acid bacteria” designates a group of Gram positive, catalase negative, non-motile, microaerophilic or anaerobic bacteria which ferment sugar (including lactose) with the production of acids including lactic acid as the predominantly produced acid, acetic acid, formic acid and propionic acid. Below are described herein preferred LAB.

The industrially most useful lactic acid bacteria are found among Lactococcus species, Streptococcus species, Enterococcus species, Lactobacillus species, Leuconostoc species, Bifidobacterium species and Pediococcus species. Accordingly, in a preferred embodiment the LAB is a LAB selected from the group consisting of these LAB.

In a preferred embodiment the LAB is a LAB selected from the group consisting of Lactococcus lactis subsp. lactis, Lactococcus lactis subsp. cremoris, Leuconostoc mesenteroides subsp. cremoris, Pediococcus pentosaceus, Lactococcus lactis subsp. lactis biovar. diacetylactis, Lactobacillus casei subsp. casei, Streptococcus thermophilus, Enterococcus faecium, Bifidobacterium animalis, Bifidobacterium lactis, Lactobacillus lactis, Lactobacillus helveticus, Lactobacillus delbrueckii subsp. bulgaricus and Lactobacillus acidophilus. Within this group, the most preferred LAB is Lactobacillus acidophilus.

The LAB culture may be a “mixed lactic acid bacteria (LAB) culture” or a “pure lactic acid bacteria (LAB) culture”.

The term “mixed lactic acid bacteria (LAB) culture” denotes a mixed culture that comprises two or more different LAB species. The term a “pure lactic acid bacteria (LAB) culture” denotes a pure culture that comprises only a single LAB species.

Commercially highly relevant mixed cultures include:
“O-culture” comprising Lactococcus lactis subsp. lactis and Lactococcus lactis subsp. cremori;
“D-culture” comprising Lactococcus lactis subsp. lactis, Lactococcus lactis subsp. cremoris and Lactococcus lactis subsp. lactis biovar. diacetylactis;
“L-culture” comprising Lactococcus lactis subsp. lactis, Lactococcus lactis subsp. cremoris and Leuconostoc mesenteroides subsp. cremoris;
“LD-culture” comprising Lactococcus lactis subsp. lactis, Lactococcus lactis subsp. cremoris, Lactococcus lactis subsp. lactis biovar. diacetylactis and Leuconostoc mesenteroides subsp. cremoris;
“Yoghurt culture” comprising Streptococcus thermophilus and Lactobacillus delbrueckii subsp. bulgaricus; and
“Thermophil cheese culture” comprising Streptococcus thermophilus and Lactobacillus helveticus.

Accordingly, in a preferred embodiment the LAB culture is a LAB culture selected from the group consisting of these cultures.

Cell Cultures

Commercially it is generally an advantage to have a relatively concentrated culture product.

Accordingly in a preferred embodiment, a composition comprising a cell culture suspended in oil, a lipid or a wax, as described herein, has a content of viable cells of at least 10⁴ colony forming units (CFU) per g composition, more preferably a content of viable cells of at least 10⁶ colony forming units (CFU) per g composition, even more preferably a content of viable cells of at least 10⁸ colony forming units (CFU) per g composition, and most preferably a content of viable cells of at least 10¹⁰ colony forming units (CFU) per g composition.

The above mentioned content of viable cells is particularly preferred when the cell is a LAB cell.

Further, it is preferred that a composition comprising a cell culture suspended in oil, a lipid or a wax, as described herein, has a weight of the composition of at least 250 g, more preferably a weight of the composition of at least 1 kg, and most preferably a weight of the composition of at least 10 kg.

A Material Comprising Oil, Lipid or a Wax

A generally preferred requirement of a material comprising oil, lipid or a wax is that it is in a melted liquid state (not solid) at 45° C. Generally speaking the material should be in melted form at a temperature that is not significantly damaging the cell of interest during the processing according to the method as described herein.

It may be advantageous that the material has a melting point between from 20° C. to 45° C.

Further, since for many uses the cell containing composition shall be given to a human or an animal it is preferred that the oil, lipid or a wax are edible.

In a preferred embodiment the material to be mixed with the cell culture of step (a) of the method of first aspect of the invention is oil. The skilled person has numerous suitable oils, lipids or waxes at his disposal.

In a preferred embodiment the lipid is a fatty acid lipid. Preferably the lipid is a lipid selected from the group consisting of: Caprylic acid, capric acid, oleic acid, linoleic acid, arachidonic acid and Lauric acid.

In a preferred embodiment the wax is a wax selected from the group consisting of: Canauba wax, candelilla wax, microcrystalline wax, beeswax and hydrogenated vegetable oil.

In a preferred embodiment the oil is a vegetable oil, preferably a vegetable oil selected from the group consisting of: Hazelnut oil, olive oil, primrose oil, pumpkin oil, rice-bran oil, soybean oil, maize oil, coconut oil, peanut oil, paraffin oil, and sunflower oil. The oil may also be an oil selected from the group consisting of: fish oil.

Porous Particles Containing Cells

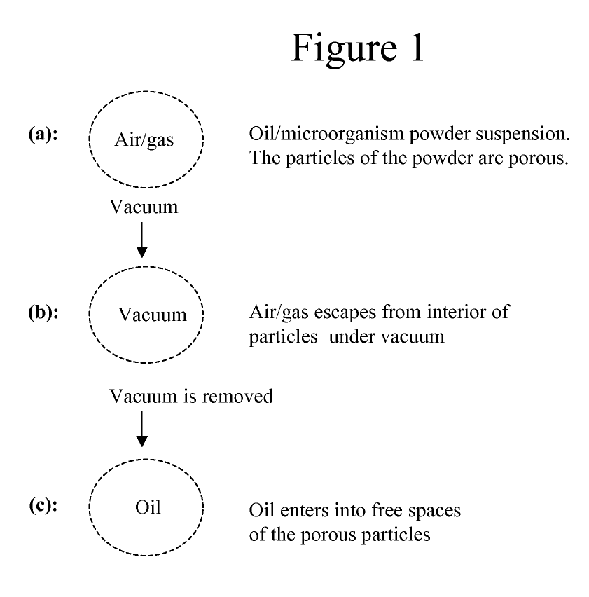

As illustrated in FIG. 1, the cell containing particles mixed with e.g. oil in accordance with step (a) of the method of the first aspect of the invention shall be porous particles. The term “porous” shall be understood as the skilled person would understand it in view of the technical objective of the method of the first aspect.

As illustrated in FIG. 1, the technical objective may be seen as to get gas out of the particles and get oil into the particles. Accordingly, it is clear that the particles must be porous in order to get the gas out and the oil into the particles.

The skilled person may easily prepare a cell culture comprising porous particles containing cells. A preferred example is a culture in the form of a powder, such as e.g. a freeze-dried powder.

As explained above, a commercially relevant microorganism culture may be a freeze-dried culture in the form of a powder. This powder comprises many individual particles. Each particle of the powder is a porous structure comprising the microorganism of interest plus other material and compounds generally derived from the previous fermentation process.

Accordingly, in a preferred embodiment the cell culture comprising porous particles containing cell is a dried culture, more preferably a dried culture in the form of a powder. Preferably, the dried culture is a freeze-dried culture, more preferably a freeze-dried culture in the form of a powder.

Preferably, the powder (such as e.g. freeze-dried powder) is milled to get powder particles with a desired particle size. A preferred particle size is less than 10 mm, more preferably less than less than 1 mm. In some applications it may be preferred that particle size is less than 500 μm, such as less than 300 μm.

For relatively larger particle size (e.g. above 1 mm) the individual particles may be termed granules and the “powder” may be termed granulate.

Step (a) of First Aspect—Mixing Porous Particles Containing Cells into e.g. Oil:

The mixing of step (a) of the method of the first aspect may be done by any suitable technique such as e.g. mechanical stirring. See e.g. working example herein for further details.

It is preferred that the mixture is stirred until no visible lumps are detected in the suspension, since this is an indication for that all the porous particles containing cells are “wetted” with the relevant material (e.g. oil). In other words, that all of the particles are covered by the relevant material (e.g. oil).

During step (a) one may optionally add further compounds of interest. This may e.g. be vitamins (e.g. tocopherol) or other compounds one could be interested in having present in the final composition.

However, at this step (a) one should preferably be careful if one wants to add the so-called viscosity enhancers (e.g. silicon dioxide). The reason is that the viscosity of the suspension could get so high that one will not get the gas efficiently removed under the subsequent vacuum step (b). Accordingly, such viscosity enhancers (e.g. silicon dioxide) should preferably be added to the suspension after removal of the vacuum (i.e. after step (c)).

To have an adequate viscosity of the suspension before the vacuum step (b) is important. However, it is easy for the skilled person to get an adequate viscosity since for instance a number of edible oils have an adequate viscosity. A suitable example is sunflower oil or the other oils mentioned above.

The viscosity range span of the commercial available oils (sunflower, olive, soy and maize) used in working example 2 herein is a viscosity range span from around 80 top around 120 cp measured on a Brookfield rheometer. This represents a useful preferred viscosity range span.

Accordingly, in a preferred embodiment the suspension of step (a) of the first aspect has, before the vacuum is created in accordance with step (b), a viscosity in the range from 1 to 1000 cp measured on a rheometer, more preferably a viscosity in the range from 25 to 200 and most preferably a viscosity in the range from 50 to 150 cp measured on a rheometer.

In step (a) the suspension preferably comprises from 5 to 40% of the cell culture (e.g. in form of a freeze-dried powder) and from 60 to 95% of the relevant material (e.g. oil). The sum of the two components cell culture and relevant material should preferably amount to at least 90% of the suspension, such as e.g. at least 95% of the suspension. In a preferred embodiment, the suspension comprises around 15 to 20% of the cell culture (e.g. in form of a freeze-dried powder) and around 75 to 80% of the relevant material (e.g. oil).

Step (b) of First Aspect—Creating a Vacuum Over the Suspension:

In step (b) a vacuum is created over the suspension in order to remove a suitable amount of the gas, present within the porous particles, from the suspension.

This may be created in different ways such as e.g. by introducing the suspension into a container suitable for creating a vacuum. For further details reference is made to working example 1 herein.

The actual pressure of the vacuum may be adjusted and optimized in relation to particular needs and requirement of the system. A generally believed adequate vacuum pressure is a pressure that is lower than 500 mbar. Generally as lower pressure as better, e.g. lower than 50 mbar or more preferably lower than 2 mbar.

As discussed above, the effect of this vacuum treatment is that at least a significant part of the gas within the particles is removed. As illustrated in working examples herein this may be seen as bubbles of gas that evaporate from the suspension.

The vacuum treatment is maintained for a suitable time. The actual chosen time period will generally depend on several factors. For instance if the suspension is stirred while under vacuum the gas escapes more rapidly and if the vacuum pressure is relatively low the gas will generally be removed faster. It is within the skilled person's knowledge to optimize this according to specific requirements of interest.

However, as a general rule one should preferably maintain the vacuum treatment until there is less than 5% of gas bubbles escaping from the suspension as compared to the initial period of vacuum treatment. More preferably, one may maintain the vacuum treatment until virtually no gas bubbles are escaping from the suspension.

A generally believed suitable time for the vacuum step (b) is a time period from 1 second to 1 hour.

It is here interesting to note that if one has a very low vacuum pressure (e.g. preferably a pressure lower than 5 mbar or more preferably lower than 1 mbar) then water vapor may escape from the suspension and bubbles will continue to be created from the product for quite a long time. This is no longer gas from the porous particles containing cells, but sublimation or desorbtion of water from the particles in the suspension.

In fact this may be seen as a further advantageous possibility of the method as described herein, since it may be seen as a “perfect” drying of the suspension. The by sublimation removed water will, as for the gas removal, create further extra “empty” pockets within the porous particles, where the relevant material (e.g. oil) can enter after vacuum removal in accordance with step (c). After vacuum removal of step (c) the suspension is generally treated in an adequate way to e.g. be packed in a suitable way (e.g. in capsules—see below).

It is difficult to do this without the suspension getting in contact with any moisture. However, since “water occupying” places have also been “occupied” with relevant material (e.g. oil) the risk of getting too much unwanted moisture incorporated into the suspension has been minimized.

Accordingly, in a preferred embodiment the vacuum pressure is so low (e.g. preferably a pressure lower than 5 mbar or more preferably lower than 1 mbar) that besides the gas also water is removed, via sublimation or desorbtion, from the suspension.

Step (c) of the First Aspect—Removing the Vacuum Over the Suspension:

In step (c) the vacuum is removed over the suspension to get an adequate pressure allowing the oil, the lipid, the wax or a mixture of these, which cover the particles, to enter into the porous particles and thereby occupy a suitable amount of the space within the particles that before the vacuum step (b) were occupied by gas.

A suitable example of an adequate pressure is a pressure around atmospheric pressure (around 1 bar).

The removal of the vacuum should preferably be done relatively quickly, in the sense that one goes from the low vacuum pressure to e.g. atmospheric pressure in a relatively short time period (e.g. within a period from instantly to 30 seconds).

When the vacuum is removed it is highly preferred that the relevant material (e.g. oil) completely covers the porous particles containing cells. To ensure this, it may be preferred to stop the stirring of the suspension just before removal of the vacuum.

After an adequate pressure (e.g. 1 bar) is established over the suspension, i.e. after or during vacuum removal, the relevant material (e.g. oil) that covers the particles enter rapidly or instantly into the porous particles. Accordingly rapidly or instantly after vacuum removal the suspension is ready for eventual further processing.

Addition of Other Compounds Such as Viscosity Enhancers:

During any steps of the method, as described herein, one may add further compounds of interest.

This may e.g. be vitamins (e.g. tocopherol) or other compounds one could be interested in having present in the final composition. Other examples of compounds of interest could be moisture scavengers such as e.g. potato starch or sucrose. Further one could e.g. add suitable cryoprotective agents.

As discussed above viscosity enhancers should preferably not be added before the vacuum step (b). However, a viscosity enhancer may preferably be added after step (c).

Accordingly, in a preferred embodiment a viscosity enhancer is added to the composition after step (c) in order to get a composition with a viscosity of interest. Preferably, it gives a composition comprising a viscosity enhancer and that has a viscosity within a range from 1000 to 100.000 cp measured on a rheometer, more preferably 2000 to 25.000 cp measured on a rheometer.

Suitable examples of viscosity enhancers include viscosity enhancers such as glycerols (eg. glycerine); glycols (e. g., polyethylene glycols, propylene glycols); plant-derived waxes (e.g., carnauba, rice, candililla), non-plant waxes (beeswax); lecithin; plant fibers; lipids; and silicas (e. g., silicon dioxide). Preferably, the viscosity enhancer is silicon dioxide.

Packing the cell composition in a suitable way:

A further possible step of the method as described herein relates to packing the cell composition in a suitable way.

The term “packing” should be understood broadly. It denotes that once the cell containing composition is obtained it should be packed in order to be provided to the consumer. It may be packed in a bottle, a tetra-pack, capsule, etc. Preferably, on the package or in corresponding marketing material is indicated what type the cell is and maybe also relevant industrial uses of it.

Preferably, a composition comprising a cell culture suspended in a relevant material (e.g. oil) as described herein is packed into a suitable capsule. The capsule may e.g. be based on an “anaerobic encapsulation system” as described in section [0055] to [0059] of WO2004/028460.

Use of a Cell Containing Composition as Described Herein:

Generally the specific preferred industrial use of a cell containing composition as described herein would normally depend on the specific characteristics of the cell in question.

If the cell is a LAB a relevant use may be to use the composition as a dairy starter culture to e.g. make cheese, yogurt or other relevant food products.

A preferred use relates to spraying the cell containing composition as described herein onto the surface of cereals. See working example 3 herein for further details.

Alternatively, it may be given to a human, an animal or a fish for health improving purposes. This is generally most relevant if the cell has probiotic properties and is particularly relevant when the cell is a probiotic LAB cell.

Novel Compositions as Such—Separate Aspect of Invention:

In working examples herein several different lactic acid bacteria compositions have been analyzed and preferred ones have been identified. These preferred compositions are characterized by having a specific novel composition of different ingredients. These novel compositions represent therefore herein a separate novel aspect of the invention.

Accordingly, a separate novel aspect of the invention relates to a composition comprising a lactic acid bacteria culture suspended in oil and characterized by that the composition comprises following ingredients:

lactic acid bacteria culture from 15 to 35% w/w

vegetable oil from 60 to 85% w/w

potato starch, from 5 to 25% w/w; and

silicon dioxide from 1 to 5% w/w.

Preferred specific amounts of the ingredients are:

lactic acid bacteria culture from 15 to 25% w/w

vegetable oil from 70 to 80% w/w

potato starch, from 5 to 15% w/w; and

silicon dioxide from 2 to 4% w/w.

Generally if one includes relatively high amounts of potato starch then one may have relatively low amounts of silicon dioxide.

Preferably the composition also comprises tocopherol, preferably from 1 to 5% w/w.

Preferably the bacteria culture is a dried culture, more preferably a dried culture in powder form. Most preferably it is a freeze-dried culture.

Preferably the vegetable oil is sunflower oil.

Preferably, the lactic acid bacteria are Lactobacillus acidophilus, Lactobacillus casei subsp. casei, Bifidobacterium lactis.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### Examples

Materials and Methods

Milling:

The freeze-dried granulate/culture was milled in a Quadro Comil 194, Screen 045R 031/37 (circular holes, Ø1.350 mm), at rotation speed: 1400 rpm. The powder was sieved to <180 μm.

### Example 1

### Test of Effect of Vacuum Pre-Treatment—Cell was L. Acidophilus

This example demonstrates the stability improvement obtained by using the vacuum pre-treatment step as described herein.

The cell culture was freeze-dried L. acidophilus (LA-5™) LAK formulation (item No. 501082). It is a commercially available Lactobacillus acidophilus culture obtainable from Chr. Hansen A/S, Denmark. LAK was milled and sieved to <180 μm. Freeze-dried to Aw 0.040.

LAVA formulation 18

| | | | |
|---|---|---|---|
| LAK, item No. 501082 | Chr. Hansen | 100.0 g | 19.6% w/w |
| Sunflower oil, EU-pharma | Bressmer & Francke | 385.0 g | 75.3% w/w |
| Tocopherol | | 10.0 g | 2.0% w/w |
| Aerosil 200 Pharma | Degussa | 16.0 g | 3.1% w/w |

Aerosil 200 Pharma is a commercial silicon dioxide product.

LAVA 18 was made in the following way:

(a): LAK and Sunflower oil were mixed mechanically until no visible lumps were detectable in order to get a suspension.

(b): A vacuum was created over the suspension by putting it into a vacuum pump and creating a pressure of less than 1 mbar. The vacuum was maintained until the gas bubbles stopped coming from the product.

(c): The vacuum was rapidly removed to get atmospheric pressure (1 bar) over the suspension.

(d): Finally Aerosil 200 was added to get a desired viscosity. Tocopherol was also added.

LAVA Formulation 20

The ingredients and amounts of these were identical to LAVA 18. It was made by the same method as for LAVA 18 except that the vacuum pre-treatment step was NOT included.

The formulations were stored at different storage conditions and in different packaging in order to evaluate the accelerated stability. Accelerated stability was measured after 3 weeks of storage and the cell counts were compared to the reference sample stored at 5° C. The results are shown in table 1 below.

TABLE 1
Comparison of the results obtained from LAVA 18, which is the basic
formulation manufactured with pre-treatment and LAVA 20, which is
the identical formulation manufactured without pre-treatment.
Survival after 21 days (%) relative to reference:

| | LAVA | LAVA |
|---|---|---|
| Storage condition | 18 | 20 |
| Reference: 5° C. (glass bottles) | 100 | 100 |
| 30° C./65% RH, HDPE container | 99 | 89 |
| 5° C./76% RH, Petri-dish | 96 | 94 |
| 30° C./30% RH, Petri-dish | 66 | 57 |

Conclusion:

These results clearly demonstrate that the vacuum treated LAVA 18 composition has a significantly improved stability as compared to the LAVA 20 control composition (without the vacuum treatment).

### Example 2

### Effect of Different Ingredients in Vacuum Treated Composition

The purpose of this experiment was to analyze the stability effect of different ingredients. LAVA 18 was used as reference. Accordingly, for other LAVA formulations mentioned below are only mentioned the difference to LAVA 18.

Effect of Antioxidant Tocopherol

TABLE 2
Comparison of the results obtained on LAVA 18 including 2%
tocopherol and LAVA 19 without tocopherol and in addition LAVA
20 manufactured with tocopherol but without pre-treatment and
LAVA 21 without tocopherol and pre-treatment
Survival after 21 days (%) relative to reference:

| | LAVA | LAVA | LAVA | LAVA |
|---|---|---|---|---|
| Storage condition | 18 | 19 | 20 | 21 |
| Reference: 5° C. (glass bottles) | 100 | 100 | 100 | 100 |
| 30° C./65% RH, HDPE container | 99 | 100 | 89 | 98 |
| 5° C./76% RH, Petri-dish | 96 | 98 | 94 | 91 |
| 30° C./30% RH, Petri-dish | 66 | 69 | 57 | 61 |

Comment:

The results of table 2 show that addition of Tocopherol has limited effect within the period tested. The significant stability improvement obtained by the vacuum treatment was confirmed.

Effect of Adding a Moisture Scavenger

TABLE 3
Two moisture scavengers were tested; Potato starch and Sucrose.
Survival after 21 days (%) relative to reference:

| | LAVA | LAVA | LAVA | LAVA |
|---|---|---|---|---|
| Storage condition | 18 | 24 | 25 | 26 |
| Reference: 5° C. (glass bottles) | 100 | 100 | 100 | 100 |
| 30° C./65% RH, HDPE container | 99 | 98 | 81* | 99 |
| 5° C./76% RH, Petri-dish | 96 | 94 | 94 | 96 |
| 30° C./30% RH, Petri-dish | 66 | 79 | 67 | 75 |

LAVA 18 as the reference. LAVA 24 containing 50 g potato starch and 14 g Aerosil, LAVA 25 containing 50 g sucrose and 12 g Aerosil and LAVA 26 containing 25 g potato starch, 25 g sucrose and 13 g Aerosil
*High standard deviation

Comment:

The results of table 3 show there is an effect of potato starch as the stability of LAVA 24 and LAVA 26 is significantly better at the storage conditions with the large surface (30C/30% RH, Petri-dish).

Test of Different Oils.

TABLE 4
LAVA 32 contains olive oils extra virgin, LAVA 33
contains soy been oil and LAVA 34 contain maize oil.
Survival after 21 days (%) relative to reference:

| | LAVA | LAVA | LAVA |
|---|---|---|---|
| Storage condition | 32 | 33 | 34 |
| Reference: 5° C. (glass bottles) | 100 | 100 | 100 |
| 30° C./65% RH, HDPE container | 100 | 99 | 99 |
| 5° C./76% RH, Petri-dish | 99 | 95 | 95 |
| 30° C./30% RH, Petri-dish | 78 | 70 | 74 |

Comment:

The results of table 4 show different oils like olive oil, soy been oil and maize oil can be used as base of the LAVA formulation. All three oils work well in the formulation.

Conclusion:

In summary the overall results of this example 2 show that a preferred oil composition a composition that overall comprises following ingredient:
lactic acid bacteria culture from 15 to 25% w/w
vegetable oil from 70 to 80% w/w
potato starch, from 5 to 15% w/w; and
silicon dioxide from 2 to 4% w/w.

A very preferred example of such a composition is LAVA 35.

TABLE 5
Stability of the preferred composition LAVA 35
Survival after 21 days (%) relative to reference:

| | LAVA |
|---|---|
| Storage condition | 35 |
| Reference: 5° C. (glass bottles) | 100 |
| 5° C./76% RH, Petri-dish | 90 |
| 30° C./30% RH, HDPE container with capsules | 77 |

Comment:

The results of table 5 show a god stability of the LAVA formulation even when stored in HDPE container with addition of gelatin capsules containing large amounts of water.

### Example 3

### Cereal Application

To evaluate if the LAVA formulation is well working on cereals the following study was set up.

LAVA 17 was used as the formulation. It was identical to LAVA 18 except that a different sunflower oil (sunflower oil BECEL from IRMA, Denmark) was used. LAVA 17 was made in the same way as LAVA 18 including the vacuum step.

The LAVA 18 formulation was applied in a small layer on the surface of cereals by spraying or brushing.

Cereals:

Nestlé Fitness flakes L42860819 (13 09:59 expiry date Dec. 10, 2005)

Approximately 10 grams of the flakes were weighed onto a Petri disk standing on the balance. A tiny layer of the LAVA 17 was applied onto the surface of the flakes. The total weight of flakes and LAVA were approximately 11 grams.

The weight of the flakes and the total weight were recorded for each sample.

The flakes with the LAVA were stored in aluminum bags at 30° C. for 3 weeks and also in a climate chamber in open bags at 30° C./30% RH for 3 weeks. The storage survival was calculated relatively to flakes stored at 5° C.

The CFU/g was measured in accordance with the DK-PIM-ins-034/035/036

TABLE 6
Flakes with LAVA stored at different storage conditions
Survival after 21 days of storage

| CPU per gram LAVA | Average | Survival |
|---|---|---|
| LAVA +5° C. | 2.11E+10 | 100% |
| LAVA +30° C. | 2.16E+10 | 102% |
| LAVA +30° C.; 30% RH | 1.16E+10 | 55% |

Comment:

From these results it shows that it is possible to apply the LAVA formulation on the cereals and that the probiotic bacteria are very storage stable.

### Example 4

### Capsule Application

Example to demonstrate the use of LAVA formulation in hard capsules.

LAVA formulation 18 containing Freeze-dried L. acidophilus (LA-5™) LAK formulation (item No. 501082) was prepared as described in example 1.

The LAVA formulation was filled into hard gelatin capsules, Coni-Snap size 3, Capsugel or HPMC capsules, size 3, Shionogi Qualicaps S.A. Capsules were stored at different storage conditions and cell count was evaluated after 3 weeks of storage.

TABLE 7
Two types of capsules containing LAVA stored
at different storage conditions
Survival after 21 days (%) relative to reference:

| | LAVA 18 capsules | |
|---|---|---|
| Storage condition | Gelatin capsules | HPMC capsules |
| Reference: 5° C. | 100 | 100 |
| (in aluminum foil bag) | | |
| 30 C./30% RH (aluminum foil bag) | 92 | 98 |
| 30 C./30% RH (HDPE container) | 64 | 63 |

Comment:

The results of table 7 show a good stability of LAVA in capsules where the surface area is very large even when stored in HDPE container.

### Example 5

### Test of Effect of Vacuum Pre-Treatment—Cell was Bifidobacterium Lactis

This example corresponds to example 1, in the sense that two formulations were made; one that included the vacuum pre-treatment step and a control without the vacuum treatment.

The cell was Bifidobacterium lactis (BB-12®). This is a commercially available (Chr. Hansen A/S, Denmark) freeze-dried culture.

The stability results, essentially made as described in example 1, demonstrated a significantly improved stability of BB-12® for the vacuum treated formulation.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

WO2004/028460 (Probiohealth LLC)

All references cited in this patent document are hereby incorporated herein in their entirety by reference.

1. A method for making a composition comprising a cell culture suspended in oil, a lipid, a wax or a mixture of those comprising the following steps:
(a): mixing a cell culture comprising porous particles containing cells with a material comprising oil, a lipid, a wax or a mixture of those;
(b): creating a vacuum over the suspension; and
(c): removing the vacuum over the suspension.

2. The method of claim 1 for making a composition comprising a cell culture suspended in oil, a lipid, a wax or a mixture of those comprising following steps:
(a): mixing a cell culture comprising porous particles containing cells into a material comprising oil, a lipid, a wax or a mixture of those to get a suspension;
(b): creating a vacuum over the suspension in order to remove a suitable amount of the gas, present within the porous particles, from the suspension; and
(c): removing the vacuum over the suspension to get an adequate pressure allowing the oil, the lipid, the wax or a mixture of these, that cover the particles, to enter into the porous particles and thereby occupy a suitable amount of the space within the particles that before the vacuum step (b) were occupied by gas;
to get a composition comprising a cell culture suspended in oil, a lipid, a wax or a mixture of those characterized by that a significant amount of the space within the porous particles containing cells that before the vacuum step (b) were occupied by gas are occupied by oil, a lipid, a wax or a mixture of those.

3. The method of claim 1, wherein the cell is a lactic acid bacteria (LAB), preferably wherein the lactic acid bacteria is Lactobacillus acidophilus, and wherein the composition comprising a cell culture has a content of viable cells of at least 10⁶ colony forming units (CFU) per g composition.

4. The method of claim 1, wherein the cell culture comprising porous particles containing cells of step (a) is a freeze-dried culture in the form of a powder.

5. The method of claim 1, wherein the material to be mixed with the cell culture of step (a) is oil, preferably a vegetable oil selected from the group consisting of: hazelnut oil, olive oil, primrose oil, pumpkin oil, rice-bran oil, soybean oil, maize oil and sunflower oil.

6. The method of claim 1, wherein the mixture of step (a) is stirred until no visible lumps are detected in the suspension.

7. The method of claim 1, wherein the suspension of step (a) comprises from 5 to 40% of the cell culture and from 60 to 95% of the relevant material and wherein the sum of the two components cell culture and relevant material amount to at least 95% of the suspension.

8. The method of claim 1, wherein the vacuum treatment of step (b) is maintained until there is virtually no gas bubbles escaping from the suspension.

9. The method of claim 1, wherein a viscosity enhancer is added to the composition after the vacuum removal step (c) in order to get a composition with a viscosity of interests.

10. The method of claim 1, wherein the composition comprising the cell culture obtained after step (c) is packed into a suitable capsule.

11. The method of claim 1, wherein the composition comprising the cell culture obtained after step (c) is sprayed onto the surface of cereals.

12. A composition obtainable by a method of claim 1.

13. The composition of claim 12, which comprises a Bifidobacterium strain and a vegetable oil.

14. The composition of claim 1, which comprises the strain BB-12® and a vegetable oil selected from the group consisting of: hazelnut oil, olive oil, primrose oil, pumpkin oil, rice-bran oil, soybean oil, maize oil and sunflower oil.

15. A composition obtainable by the method comprising the following steps:
(a): providing a suspension containing (i) cells, (ii) porous particles, and (iii) an oil, a lipid, or a wax;
(b): creating a vacuum over the suspension; and
(c): removing the vacuum over the suspension.

16. A composition of claim 1, which is encapsulated.

17. A food product comprising a composition of claim 1.

18. A food or feed additive comprising a composition of claim 1.
